(19) United States
(12) Patent Application Publication
(10) Pub. No.: US 2006/0110733 A1
(43) Pub. Date: May 25, 2006

(54) Coded nucleic acid carriers

(76) Inventors: Brendan James Toohey, Springbank Victoria (AU); Karl Frederick Poetter, Northcote Victoria (AU)

Correspondence Address: SCULLY, SCOTT, MURPHY & PRESSER, 400 GARDEN CITY PLAZA, SUITE 300, GARDEN CITY, NY, 11530, US

(21) Appl. No.: 10/525,356

(22) PCT Filed: Aug. 22, 2003
(86) PCT No.: PCT/AU03/01077

§ 371 Date: Dec. 2, 2005

(30) Foreign Application Priority Data
Aug. 23, 2002 (AU) 2002950953

(51) Int. Cl. C12Q 1/68; G06F 19/00

(52) U.S. Cl. 435/6; 702/20

(57) ABSTRACT

The present invention relates generally to coded solid or semi-solid nucleic acid carriers for use in multiplexing solid phase nucleic acid-based reactions. The use of coded carriers facilities multiplexing due to the ability to deconvolute multiple nucleic acid-based events and to correlate those to particular experiments. The present invention further provides a method for identifying a nucleic acid molecule having a defined characteristic within a population of two or more different nucleic acid molecules using coded nucleic acid carriers. Conversely, the nucleic acid can be used as the code for a particular peptide, or other chemical, bound specifically to a microsphere with a specific oligonucleotide sequence. Alternatively, the method of the present invention permits screening for molecules which interact with target nucleic acid, or other, molecules. The method and the coded carriers of the present invention enable high throughput screening of nucleic acid, or other, molecules. The method may also be automated and/or controlled by computer software.

### BACKGROUND OF THE INVENTION

1. Field of the Invention

The present invention relates generally to coded solid or semi-solid nucleic acid carriers for use in multiplexing solid phase nucleic acid-based reactions. The use of coded carriers facilitates multiplexing due to the ability to deconvolute multiple nucleic acid-based events and to correlate these to particular experiments. The present invention further provides a method for identifying a nucleic acid molecule having a defined characteristic within a population of two or more different nucleic acid molecules using coded nucleic acid carriers. Conversely, the nucleic acid can be used as the code for a particular peptide, or other chemical, bound specifically to a microsphere with a specific oligonucleotide sequence. Alternatively, the method of the present invention permits screening for molecules which interact with target nucleic acid, or other, molecules. The method and the coded carriers of the present invention enable high throughput screening of nucleic acid, or other, molecules. The method may also be automated and/or controlled by computer software.

### BACKGROUND OF THE INVENTION

Bibliographic details of references provided in the subject specification are listed at the end of the specification.

Reference to any prior art in this specification is not, and should not be taken as, an acknowledgment or any form of suggestion that this prior art forms part of the common general knowledge in any country.

The increasing sophistication of recombinant DNA technology is greatly facilitating research and development in a range of biotechnology-related industries. Of particular importance is the development of solid phase methodologies for a range of nucleic acid-based manipulations such as polymerase chain reaction (PCR), hybridization interactions and interactions between nucleic acid molecules and chemical or biological agents. Such techniques are useful inter alia for the identification of particular nucleotide sequences including polymorphisms such as single nucleotide polymorphisms (SNPs).

Solid phase nucleic acid manipulations have involved solid supports such as microtitre wells as well as microparticles. Examples of microparticles include microspheres. DNA and other chemical manipulations, such as chemical libraries, on microspheres have many advantages. However, the absence of a reliable and accurate way of multiplexing multiple experiments in a single reaction vessel is a rate limiting step in the development of high throughput systems based on microsphere technology. The potential power of microsphere technology combined with light or radiation detection systems is enormous in terms of developing high throughput screening protocols for nucleic acid molecules. For example, some flow cytometers can read and sort microspheres with fluorescent signals at rates of up to 100,00 microspheres per second.

Whilst present fluorescent signals are useful in screening for fluorescently labeled nucleic acid molecules immobilized to microspheres, they are not reliable enough to permit the required depth of multiplexing for high throughput screening.

There is a need, therefore, to develop methodologies which involve multi-dimensional analysis of nucleic acid carriers and products of nucleic acid-based reactions.

### SUMMARY OF THE INVENTION

Throughout this specification, unless the context requires otherwise, the word “comprise”, or variations such as “comprises” or “comprising”, will be understood to imply the inclusion of a stated element or integer or group of elements or integers but not the exclusion of any other element or integer or group of elements or integers.

The present invention is predicated in part on the use of codes distinctive of a solid or semi-solid phase carrier for nucleic acids or other molecules to deconvolute multiplexed reactions occurring on a population of two or more carriers. A carrier in this context may be any solid or semi-solid substrate for nucleic acid molecules. Carriers contemplated herein include inter alia microspheres, beads, cubes or ovoids. Preferably, the carrier is a microsphere or other microparticle or nanoparticle.

The carriers and methods of the present invention allows for the simultaneous testing and sorting of many samples in which events which “pass” the test by fluorescence measurement are determined from analysis of a second code co-bound to each microsphere. Conversely, this method allows the screening of small chemicals bound to DNA coded beads, by reacting the beads with a fluorescent substrate, sorting the successes by fluorescence and then determining the chemical sample by determination of the DNA code. This method should enhance the sensistivity of the test because the DNA moiety from single microspheres can be amplified by DNA or RNA polymerisation techniques.

Consequently, the present invention provides a carrier on which a nucleic acid-based reaction may occur wherein the carrier displays a code distinctive of the carrier. A code is preferably an attribute incorporated or associated with the carrier. Examples of codes include peptides, polypeptides or proteins or other polymers, carbohydrates, phospholipids, nucleic acids, antibodies or any other feature which assists in distinguishing one carrier from another. In a preferred embodiment, the code is distinguishable based on mass. In this case, mass spectrometry is a convenient means of sorting the carriers. Where the code molecule is a nucleic acid, methods for direct or indirect determination of the nucleotide sequence of the nucleic acid code molecule are particularly useful.

The nucleic acid-based reactions generally involve an amplification or polymerization reaction and/or hybridization interaction to incorporate a reporter molecule such as a fluorescent marker, such as a fluorophore. In addition, nucleic acid-based interactions include the binding or association of chemical or biological agents to nucleic acid molecules.

When the subject molecule is not a nucleic acid, binding of a labelled ligand to the subject molecule is contemplated as a mechanism for incorporation of a label.

Generally, after incorporation of a fluorophore-labeled nucleic acid into a nucleic acid molecule immobilized onto a carrier, or binding of a labeled ligand to a non-nucleic acid molecule there is an initial sorting based on the fluorescent marker such as by flow cytometry. This is followed by a deconvolution based on the code distinctive of the carrier. As indicated above, preferably, the code is a molecule having a distinctive mass, or nucleotide sequence. Peptide codes are particularly preferred where the subject molecule is a nucleic acid, whereas when the subject molecule is a non-nucleic acid, nucleic acid code molecules are preferred. In an alternative embodiment, labeled chemical agents such as from a chemical library or biological library are exposed to a specific agent or cell type. Beads which bind or associate or are altered by the agent or cell type are sorted by a recognizable fluorescence change in the chemical agent fixed to the bead. The chemical is then deduced by determination of the co-bound nucleic acid moiety.

In yet another embodiment, the label on the nucleic acid primer or on the putative nucleic acid binding ligand and the code are one and the same molecule.

The nucleic acid molecules are conveniently immobilized to the carrier by any means but via a universal anchoring system is particularly preferred.

In use, one aspect of the method of the present invention generally involves:-
(i) co-conjugating nucleic acid molecules and codes (e.g. peptides) to a population of carriers such that particular nucleic acid molecules are immobilized to carriers having a distinctive code;
(ii) incorporating fluorescent labels or other labels into the immobilized molecule via amplification, polymerization and/or hybridization reactions or binding agents;
(iii) conducting a first dimensional sorting of the carriers based on expression of the fluorescent or other labels; and
(iv) conducting a second dimensional sorting of the first dimensionally sorted carriers based on the carrier codes.

In this way, multiple experiments can be conducted on a larger scale.

In another aspect, the present invention resides in a method of producing a plurality of carriers including a population having detectably distinct carriers, comprising the steps of:-
(a) preparing a plurality of carriers having different codes wherein each code is associated with a respective carrier;
(b) subjecting the immobilized molecules to nucleic acid based or ligand binding reactions to enable incorporation of detectable labels into the immobilized molecules;
(c) identifying carriers having distinctive codes;
(d) identifying carriers having similar codes; and
(e) sorting the carriers having distinctive codes from the carriers having non-distinctive codes to thereby provide a plurality of carriers including a population having detectably distinct codes.

### BRIEF DESCRIPTION OF THE FIGURES

FIG. 1 is a schematic representation of the co-labelling of beads with a specific chemical. 1. Silica microsphere is silanized with 3-mercaptopropyl trimethoxysilane to yield a thiol blanket on the microsphere. 2. Thiolated microspheres are reacted with Acrydite® DNA. 3. The surface of the microsphere is a mixture of DNA attached via thioether bonds and S-S bonds. 4. S-S bonds are reduced. 5. Reactivated thiols are reacted with sulfur agent (Iodaacetamide:Fl, in this example).

FIG. 2 is a schematic representation of the co-labelling of beads with a specific chemical. 1. Silica beads are thiolated with 3-mercaptopropyl trimethoxysilane. 2. Thiolated microspheres are reacted with aminated DNA with a 5′ ethylene group. 3. DNA with amines bound covalently. 4. Amines are attacked with the compound of interest, in this example, a compound (R) with an active succinimidyl ester (SE) group.

FIG. 3 is a diagrammatic representation of a cross-section of a computer readable data storage system.

FIG. 4 is a diagrammatic representation of a cross-section of a magnetically readable data storage system.

FIG. 5 is a diagrammatic representation of a cross-section of an optically readable data storage system.

### DETAILED DESCRIPTION OF THE PREFERRED EMBODIMENTS

The present invention provides a carrier for a nucleic acid or other molecule which is coded or tagged for identification purposes. It is proposed, therefore, that multiple carriers within a population of carriers each carry a distinguishable code or carrier. A nucleic acid reaction or event or ligand binding event associated with any one carrier or group of carriers can then be grouped or pooled based on the code associated with that particular carrier or group of carriers.

The term “carrier” is used in its broadest sense to include any particular solid support or semi-solid support having surface or sub-surface chemistries appropriate for the immobilization of nucleic acid molecules and a code. Although the carrier may be any suitable size or shape, particles and in particular microparticles or nanoparticles or other bead-like particles are preferred. The shape of the particles may be spheres, cubes, rectangular prisms, pyramids, cones, ovoids, sheets or cylinders. Microspheres are particularly preferred. Generally, the surface chemistry of the particle needs to be appropriate for the formation of covalent bonds between the surface and nucleic acid or chemical entities.

The carriers of the present invention have two important attributes:-
(i) they are coded with a molecule which can distinguish one particular carrier from another; and
(ii) they carry a nucleic acid molecule or other molecule, or a chemical linking moiety capable of entering into chemical bond formation with a nucleic acid molecule.

The code or tag may be of any particular type such as distinguishable based on size, composition, mass, immunological specificity, nucleotide sequence or spectral characteristics. Codes or tags distinguishable based on mass are particularly preferred in accordance with the present invention. The preferred codes are peptides, polypeptides or proteins but any polymer or chemical agent may be employed. Furthermore, the code may be a label or a primer which is incorporated into immobilized nucleic acid material or may be the label on a chemical or biological agent which binds to a target immobilized nucleic acid molecule.

In an alternate embodiment, when the molecule of interest (also referred to herein as the interactive molecule or immobilized molecule) is a non-nucleic acid molecule the code molecule may be a nucleic acid. In accordance with this embodiment, the code molecule may be distinguished on the basis of the nucleotide sequence of the code molecule.

Accordingly, the present invention provides a carrier comprising a nucleic acid molecule or other molecule immobilized thereto or a chemical moiety capable of entering into chemical bond formation with a nucleic acid molecule, which carrier carries means for distinguishing or identifying that particular carrier from another carrier.

More particularly, the present invention contemplates a population of carriers each comprising a nucleic acid, or other molecule, immobilized thereon or a chemical moiety capable of chemical bond formation with a nucleic acid molecule and wherein each carrier or group of carriers within the population carry means to distinguish individual carriers or group of carriers from one another.

Preferably, the carriers are microparticles or nanoparticles such as microspheres.

Preferably, the means for identifying or distinguishing the carriers is by the incorporation or immobilization of a polymer or chemical entity having a characteristic mass or nucleotide sequence. A chemical moiety includes a nucleic acid molecule to which a second nucleic acid molecule hybridizes or ligates.

Reference to a polymer includes a peptide, polypeptide or protein. Peptide codes are particularly preferred due to the ability to generate a population of peptides each having a characteristic mass identifiable by mass spectrometry. The present invention extends, however, to any form of code having a distinctive chemical attribute such as size, mass, spectral characteristic, ligand specificity or immunological profile.

The carriers may comprise any solid material capable of providing a base for nucleic acid-based or ligand binding based reactions. For example, the carriers may be polymeric supports such as polymeric beads, which are preferably formed form polystyrene cross-linked with 1-5% divinylbenzene. Polymeric beads may also be formed from hexamethylenediamine-polyacryl resins and related polymers, poly[N-(2-(4-hydroxylphenyl)ethyl)] acrylamide (i.e. one Q), silica, cellulose beads, polystyrene beads, poly(halomethylstyrene) beads, poly(halostyrene beads, poly(acetoxystyrene) beads, latex beads, grafted copolymer beads such as polyethylene glycol/polystyrene, porous silicates, for example, controlled pore-glass beads, polyacrylamide beads, for example, poly(acryloylsarcosine methyl ester) beads, dimethylacrylamide beads optionally cross-linked with N,N′-bis-acrylolyl ethylene diamine, glass particles coated with a hydrophobic polymer inclusive of cross-linked polystyrene or a fluorinated ethylene polymer which provides a material having a rigid or semi-rigid surface, poly(N-acryloylpyrrolidine) resins, Wang (trade mark) resins, Pam resins, Merrified (trade mark) resins, PAP and SPARE polyamide resins, polyethylene functionalized with acrylic acid, kieselguhr/polyamide (Pepsyn K), polyHipe (trade mark), PS/polydimethylacrylamide copolymers, CPG, PS macrobeads and Tentagel (trade mark), PEG-PS/DVB copolymers.

It will also be appreciated that the polymeric beads may be replaced by other suitable supports such as pins or chips as is known in the art, e.g. as discussed in Gordon el al., J. Med. Chem. 37(10): 1385-1401, 1994. The beads may also comprise pellets, discs, capillaries, hollow fibres or needles as is known in the art. Reference may also be made to International Patent Publication No. WO 93/06121 which describes a broad range of supports that may constitute carriers for use in the present invention. By way of example, these carriers may be formed from appropriate materials inclusive of latex, glass, gold or other colloidal metal particles and the like. Reference may also be made to International Patent Publication Nos. WO 95/25737 or WO 97/15390 which disclose examples of suitable carriers.

A plurality of carriers according to the invention may be prepared by any suitable method. Preferably, when colloidal particles including polymeric and ceramic particles are used as carriers, the colloid dispersion of such carrier is stabilized. Exemplary methods imparting colloidal stabilization are described, for example, in Hunter (“Foundation of Colloid Science”, Oxford University Press, Melbourne) and Napper (“Polymeric stabilization of Colloidal Dispersions”, Academic Press, London). In this regard, the most widely exploited effect of non-ionic polymers on colloid stability is steric stabilization, in which stability is imparted by polymer molecules that are absorbed onto, or attached to, the surface of the colloid particles. Persons of skill in the art will recognize that it is possible to impart stability by combinations of different stabilization mechanisms, e.g. surface charge on the particles can impact colloidal stability via electrostatic stabilization and an attached polyelectrolyte can impart stability by a combination of electrostatic and steric mechanisms (electrosteric stabilization). A polymer that is in free solution can also influence colloid stability. Stabilization by free polymer is well documented (Napper, 1983, supra) and is called “depletion stabilization”.

Steric stabilization of colloid dispersions may be employed. In this regard, steric stabilization is widely exploited because it offers several distinct advantages over electrostatic stabilization. For example, one advantage is that aqueous sterically stabilized dispersions are comparatively insensitive to the presence of electrolytes because the dimensions of non-ionic chains vary relatively little with the electrolyte concentration. This contrasts sharply with the spatial extensions of electrical double layers, which are strongly dependent upon the ionic strength. It is apparent that at ionic strengths greater than ca. [10⁻² mol dm⁻³, electrical double layer thickness have shrunk to such an extent that the electrostatic repulsion may no longer outweigh the van der Waals attraction. This accounts for the coagulation of electrostatically stabilized dispersions on the addition of electrolyte. Another advantage is that it is equally effective in both aqueous and non-aqueous dispersion media. This contrasts with electrostatic stabilization, which is relatively ineffective in non-polar dispersion media. In addition, steric stabilization is equally effective at both high and low volume fractions of the dispersed phase; the high volume fraction dispersions displaying relatively low viscosities. Other advantages of sterically stabilized dispersions include good freeze-thaw stability, which can be a desirable attribute in some applications and the ability to be flocculated reversibly, which is less common with electrostatically stabilized dispersions.

The polymeric microparticle can be prepared from a variety of polymerizable monomers including styrenes, acrylates and unsaturated chlorides, esters, acetates, amides and alcohols, including but not limited to polystyrene (including high density polystyrene latexes such as brominated polystyrene), polymethylmethacrylate and other polyacrylic acids, polyacrylonitrile, polyacrylamide, polyacrolein, polydimethylsiloxane, polybutadiene, polyisoprene, polyurethane, polyvinylacetate, polyvinylcyhloride, polyvinylpyridine, polyvinylbenzylchoride, polyvinyltoluene, polyvinylidenechloride and polydivinylbenzene. The microparticles may be prepared from styrene monomers. Ceramic microparticles may be comprised of silica, alumina, titania or any other suitable transparent material. Preferably, silica particles are employed. A suitable method of making silica microparticles is described, for example, in “The Colloid Chemistry of Silica and Silicates” (Cornell University Press) by Ralph K. Der, 1955 and U.S. Pat. No. 5,439,624, the disclosures of which are incorporated herein by reference.

Microparticles may also be prepared comprising different polymeric materials and/or different ceramic materials. For example, such microparticles may comprise a plurality of layers of one or more different polymers as, for example, described in Caruso et al., J. Am. Chem. Soc. 120: 8523-8524, 1998. Polymeric particles of this type may be prepared having different refractive indices or opacities, which may be used as detectable attributes according to the present invention. Alternatively, microparticles may comprise a plurality of layers, preferably composite multi-layers, of ceramic materials as, for example, described in van Blaadern et al. Langmuir 8: 2921-2931, 1992, which is incorporated herein by reference. The atomic ratio of different ceramic materials may be used as a detectable and/or quantifiable attribute of the invention. In this regard, reference may be made to U.S. Pat. No. 5,439,624, which discloses measurement of Si/Al ratio of particles, by wavelength dispersive spectroscopy.

In a particularly preferred embodiment, the carrier is a microsphere and the code is a peptide.

In an alternate preferred embodiment, the immobilized molecule is a non nucleic acid molecule, the carrier is a microsphere and the code is a nucleic acid molecule.

Accordingly, another aspect of the present invention provides a population of microspheres each or a group of which comprises a peptide having a characteristic mass and a nucleic acid molecule and/or a chemical moiety capable of entering into chemical bond formation with a nucleic acid molecule.

The coded or labeled carriers may be used in any number of techniques. The following description of the use in relation to the identification of a particular nucleotide sequence based on PCR-mediated incorporation of a labeled primer is one of a host of techniques for which the mass coded carriers are useful. However, this description in no way limits the invention to the particular methods described.

A population of nucleic acid molecules are immobilized to a carrier either directly or via a oligonucleotide or other chemical moiety capable of entering into chemical bond formation with the nucleic acid molecule. Generally, there is a population of carriers such as microspheres each or a group of which comprises nucleic acid molecules to be assessed. Each or a group of carriers is coded with a peptide having a defined mass characteristic of that particular peptide relative lo another carrier or group of carriers.

Although not wishing to limit the invention to any one mode of code identification, the use of mass spectrometry to identify and/or distinguish the peptide is particularly preferred.

PCR is conducted using pairs of primers which are generally with a different reporter molecule capable of giving a distinguishable signal. The use of fluorophores is particularly useful in the practice of the present invention. Examples of suitable fluorophores may be selected from the list given in Table 1. Other labels include luminescence and phosphorescence as well as infrared dyes.

| Probe | Ex¹ (nm) | Em¹ (nm) |
|---|---|---|
| Reactive and conjugated probes | | |
| Hydroxycoumarin | 325 | 386 |
| Aminocoumarin | 350 | 455 |
| Methoxycoumarin | 360 | 410 |
| Cascade Blue | 375; 400 | 423 |
| Lucifer Yellow | 425 | 528 |
| NBD | 466 | 539 |
| R-Phycoerythrin (PE) | 480; 565 | 578 |
| PE-Cy5 conjugates | 480; 565; 650 | 670 |
| PE-Cy7 conjugates | 480: 565; 743 | 767 |
| APC-Cy7 conjugates | 650; 755 | 767 |
| Red 613 | 480; 565 | 613 |
| Fluorescein | 495 | 519 |
| FluorX | 494 | 520 |
| BODIPY-FL | 503 | 512 |
| TRITC | 547 | 574 |
| X-Rhodamine | 570 | 576 |
| Lissarnine Rhodamine B | 570 | 590 |
| PerCP | 490 | 675 |
| Texas Red | 589 | 615 |
| Allophycocyanin (APC) | 650 | 660 |
| TruRed | 490, 675 | 695 |
| Alexa Fluor 350 | 346 | 445 |
| Alexa Fluor 430 | 430 | 545 |
| Alexa Fluor 488 | 494 | 517 |
| Alexa Fluor 532 | 530 | 555 |
| Alexa Fluor 546 | 556 | 573 |
| Alexa Fluor 555 | 556 | 573 |
| Alexa Fluor 568 | 578 | 603 |
| Alexa Fluor 594 | 590 | 617 |
| Alexa Fluor 633 | 621 | 639 |
| Alexa Fluor 647 | 650 | 688 |
| Alexa Fluor 660 | 663 | 690 |
| Alexa Fluor 680 | 679 | 702 |
| Alexa Fluor 700 | 696 | 719 |
| Alexa Fluor 750 | 752 | 779 |
| Cy2 | 489 | 506 |
| Cy3 | (512); 550 | 570; (615) |
| Cy3,5 | 581 | 596; (640) |
| Cy5 | (625); 650 | 670 |
| Cy5,5 | 675 | 694 |
| Cy7 | 743 | 767 |
| Nucleic acid probes | | |
| Hoeschst 33342 | 343 | 483 |
| DAPI | 345 | 455 |
| Hoechst 33258 | 345 | 478 |
| SYTOX Blue | 431 | 480 |
| Chromomycin A3 | 445 | 575 |
| Michramycan | 445 | 575 |
| YOYO-1 | 491 | 509 |
| SYTOX Green | 504 | 523 |
| SYTOX Orange | 547 | 570 |
| Ethidium Bormide | 493 | 620 |
| 7-AAD | 546 | 647 |
| Acridine Orange | 503 | 530/640 |
| TOTO-1, TO-PRO-1 | 509 | 533 |
| Thiazole Orange | 510 | 530 |
| Propidium Iodide (PI) | 536 | 617 |
| TOTO-3, TO-PRO-3 | 642 | 661 |
| LDS 751 | 543; 590 | 712; 607 |
| Cell function probes | | |
| Indo-1 | 361/330 | 490/405 |
| Fluo-3 | 506 | 526 |
| DCFH | 505 | 535 |
| DHR | 505 | 534 |
| SNARF | 548/579 | 587/635 |
| Fluorescent Proteins | | |
| Y66F | 360 | 508 |
| Y66H | 360 | 442 |
| EBEP | 380 | 440 |
| Wild-type | 396, 475 | 50, 503 |
| GFPuv | 385 | 508 |
| ECFP | 434 | 477 |
| Y66W | 436 | 485 |
| S65A | 471 | 504 |
| S65C | 479 | 507 |
| S65L | 484 | 510 |
| S65T | 488 | 511 |
| EGFP | 489 | 508 |
| EYFP | 514 | 527 |
| DsRed | 558 | 583 |
| Other probes | | |
| Monochlorobimane | 380 | 461 |
| Calcein | 496 | 517 |

¹Ex: Peak excitation wavelength (nm)
²Em: Peak emission wavelength (nm)

Any suitable method of analyzing fluorescence emission is encompassed by the present invention. In this regard, the invention contemplates techniques including but not restricted to 2-photon and 3-photon time resolved fluorescence spectroscopy as, for example, disclosed by Lakowicz et al. (Biophys; J. 72; 567, 1997, incorporated herein by reference), fluorescence lifetime imaging as, for example, disclosed by Eriksson et al. (Biophys. J. 2; 64, 1993, incorporated herein by reference) and fluorescence resonance energy transfer as, for example, disclosed by Youvan et al. (Biotechnology et elia 3:1-18, 1997).

Luminescence and phosphorescence may result respectively from a suitable luminescent or phosphorescent label as is known in the art. Any optical means of identifying such label may be used in this regard.

Infrared radiation may result from a suitable infrared dye. Exemplary infrared dyes that may be employed in the invention include but are not limited to those disclosed in Lewis et al. (Dyes Pigm. 42(2): 197, 1999), Tawa et al. (Mater. Res. Soc. Symp. Proc. 488 [Electrical, Optical and Magnetic Properties of Organic Solid-State Materials IV], 885-890), Daneshvar et al. (J. Immunol. Methods 226(1-2): 119-128, 1999), Rapaport et al. (Appl. Phys. Lett. 74(3): 329-331, 1999) and Durig et al. (J. Raman Spectrosc. 24(5): 281-285, 1993), which are incorporated herein by reference. Any suitable infrared spectroscopic method may be employed to interrogate the infrared dye. For instance, fourier transform infrared spectroscopy as, for example, described by Rahman et al. (J. Org. Chem. 63: 6196, 1998) may be used in this regard.

Suitably, electromagnetic scattering may result from diffraction, reflection, polarization or refraction of the incident electromagnetic radiation including light and X-rays. In this regard, the carriers may be formed of different materials to provide a set of carriers with varying scattering properties such as different refractive indexes as, for example, described supra. Any suitable art recognized method of detecting and/or quantifying electromagnetic scatter may be employed. In this regard, the invention also contemplates methods employing contrast variation in light scattering as, for example, described in van Helden and Vrij (Journal of Colloidal and Interface Science 76: 419-433, 1980).

The fluorescence emission may result from excitation of one or more fluorescent markers attached to, or contained within, the carrier. In the case of two or more fluorescent markers being utilized, the markers may be the same wherein the markers contain varying amounts of a fluorophore and are, therefore, intensity-differentiated. Alternatively, the markers may be different wherein they are present in a ratio of 1:1 or varying ratios. Reference may be made in this regard to Yamashita et al. (International Patent Publication No. WO 95/32425).

Exemplary fluorophores which may be used in accordance with the present invention include those discussed by Dower et al. (International Patent Publication No. WO 93/06121). Preferably, fluorescent dyes are employed. Any suitable fluorescent dye may be used for incorporation into the carrier of the invention, For example, reference may be made to U.S. Pat. Nos. 5,573,909 (Singer el al.) and 5,326,692 (Brinkley et al.) which describe a plethora of fluorescent dyes. Reference may also be made to fluorescent dyes described in U.S. Pat. Nos. 5,227,487, 5,274,113, 5,405,975, 5,433,986, 5,442,045, 5,451,663, 5,453,517, 5,459,276, 5,515,864, 5,648,270 and 5,723,218.

Multiple assays may be conducted in accordance with the present invention. Each experiment comprises particular coded carriers.

PCR is then conducted which results in incorporation of the reporter molecules. In the case of fluorophore reporter molecules, these can be readily identified using FACS sorting via a flow cytometer. At this point, the products of the nucleic acid reaction are grouped on the basis of reporter molecule and not on the basis of the particular family of carriers. This is accomplished through, for example, mass spectrometry. Individual experiments are then identified based on the carrier.

The present invention provides, therefore, a method for identifying a nucleic acid molecule immobilized to a carrier and having a defined characteristic from a population of two or more carriers with nucleic acid molecules immobilized thereon wherein each carrier comprises an identifiable code which identifies the carrier, said method comprising:
coding each carrier with said identifier code and immobilizing a nucleic acid molecule to be labeled via a nucleic acid-based reaction;
subjecting the coded carriers to a nucleic acid-based reaction to incorporate the different labels attributed to pairs of PCR primers;
sorting the coded carriers on the basis of label incorporated following the nucleic acid-based reaction; and
then identifying carriers on the basis of the code.

The present invention provides, therefore, a method of producing a plurality of carriers including a population of carriers having delectably distinct carriers, The method includes:-
a) preparing a plurality of carriers having different codes wherein each code is associated with a respective carrier;
b) subjecting the nucleic acid molecules to nucleic acid-based reactions to enable incorporation of detectable labels into the immobilized nucleic acid molecules;
c) identifying carriers having distinctive codes that are detectably and/or quantifiably deciperable or resolvable by the detection/quantification means;
d) identifying carriers having similar to non-distinctive codes; and
e) sorting carriers having distinctive codes from the carriers having non-distinctive codes to thereby provide a plurality of carriers including a population having detectably distinct codes.

As indicated above, the identification steps may be effected by use of any suitable method or apparatus for analyzing the detectable/quantifiable attributes of a carrier. Preferably, these steps are effected by flow cytometry, which typically detects optical parameters. For example, a flow cytometer may be used to determine forward scatter (which is a measure of size of a carrier), side scatter (which is sensitive to refractive index and size of a particle (see Shapiro, “Practical flow cytometry”, 3^{rd} ed. Brisbane, Wiley-Liss, 1995]) and fluorescent emission.

As is known in the art, flow cytometry is a high throughput technique which involves rapidly analyzing the physical and chemical characteristics of cells or other particles as they pass through the path of one or more laser beams while suspended in a fluid stream. As each cell or particle intercepts the laser beam, the scattered light and fluorescent light emitted by each cell or particle is detected and recorded using any suitable tracking algorithm as, for example, described hereunder.

A modern flow cytometer is able to perform these tasks up to 100,000 cells/particles s⁻¹. Through the use of an optical array of filters and dichroic mirrors, different wavelengths of fluorescent light can be separated and detected simultaneously. In addition, a number of lasers with different excitation wavelengths may be used. Hence, a variety of fluorophores can be used to target and examine, for example, intra- and extra-cellular properties of individual cells. The scattered light measurements can also classify an individual carrier's size, shape. granularity and/or complexity and, hence, belonging to a particular population of interest (Shapiro, 1995, supra).

Suitable flow cytometers which may be used in the methods of the present invention include those which measure five to nine optical parameters (see Table 2) using a single excitation laser, commonly an argon ion air-cooled laser operating at 15 mW on its 488 nm spectral line. More advanced flow cytometers are capable of using multiple excitation lasers such as a HeNe laser (633 nm) or a HeCd laser (325 nm) in addition to the argon ion laser (488 or 514 nm). Optical parameters, corresponding to different optically detectable/quantifiable attributes, for a carrier, may be measured by a flow cytometer to provide a matrix of qualitative and/or quantitative information, providing a code (or addressability in a multi-dimensional space) for the carrier.

TABLE 2
Exemplary optical parameters which may be measured by a flow
cybomerer and which were used in the present investigation.

| | | Detection angle form | Wavelength |
|---|---|---|---|
| Parameter | Acronym | incident laser beam | (nm) |
| Forward scattered light | FS | 2-5° | 488^{*} |
| Side scattered light | SS | 90° | 488^{*} |
| “Green” fluorescence | FL1 | 90° | 510-540^{†} |
| “Yellow” fluorescence | FL2 | 90° | 560-580^{†} |
| “Red” fluorescence | FL3 | 90° | >650^{#} |

^{*}using a 488 nm excitation laser
^{†} width of bandpass filter
^{#} longpass filter

For example, Biggs el al. (Cyrometry 36: 36-45, 1999) have constructed an 11-parameter flow cytometer using three excitation lasers and have demonstrated the use of nine distinguishable fluorophores in addition to forward and side scatter measurements for purposes of immunophenotyping (i.e. classifying) cells. The maximum number of parameters commercially available currently is 17: forward scatter, side scatter and three excitation lasers each with five fluorescence detectors. Whether all of the parameters can be adequately used depends heavily on the extinction coefficients, quantum yields and amount of spectral overlap between all fluorophores (Malemed et al., “Flow cytometry and sorting”, 2^{nd} Ed., New York, Wiley-Liss, 1990). However, it will be understood that the present invention is not restricted to any particular flow cytometer or any particular set of parameters. In this regard, the invention also contemplates use in place of a conventional flow cytometer, a microfabricated flow cytometer as, for example, disclosed by Fu et al. (Nature Biotechnology 17: 1109-1111, 1999).

A further advantage of flow cytometry is the ability to physically separate a cell or particle of interest from a heterogenous population of cells/particles. This is achieved through electrical or mechanical means by collecting desired cells/particles at a point downstream from the laser beam while undesired cells/particles continue to flow into a waste container. A flow cytometer with this capacity to sort is known as a “fluorescence-activated cell sorter” (FACS). Accordingly, the step of sorting in the present method of obtaining a population of detectably unique carriers may be effected by flow cytometric techniques such as by fluorescence activated cell sorting (FACS) although with respect to the present invention, FACS is more accurately “fluorescence activated carrier or solid support sorting” (see, for example, “Methods in Cell Biology” Vol. 33, Darzynkiewica, Z. and Crissman, H. A., eds., Academic Press) and Dangl and Herzenberg, J. Immunol. Methods 52: 1-14, 1982, both incorporated herein by reference.

Any suitable algorithm may be employed to track and/or sort individual detectably unique carriers. Preferably, a real-time algorithm is employed. For example, the real-time algorithm may divide a “parameter space” into smaller pre-defined grid spaces wherein all the grid spaces are registered empty. As carriers from a sample population pass through the flow cytometer in a single file, the codes belonging to each carrier will correspond to a particular grid space. Two possible outcomes can then occur:-
(i) if the grid space is registered empty, the carrier is sorted and collected by the flow cytometer and the grid space is registered full; or
(ii) if the grid space is already registered full, the carrier is rejected.

Suitably, the step of sorting is characterized in that the population of detectably distinct carriers constitutes at least about 50%, preferably at least about 70%, more preferably at least about 90% and more preferably at least about 95% of the plurality of carriers.

The present invention extends to the use of the coded carriers to detect nucleic acid binding chemical or biological agents. In one embodiment, the present invention provides a method for detecting a nucleic acid binding agent comprising:
preparing nucleic acid molecules immobilized to a carrier wherein each carrier or group of carriers is coded with an identification code;
contacting the carrier with putative nucleic acid-binding agents each labeled with a reporter molecule;
conducting a first dimensional sorting based on the reporter molecule of an agent if bound to the nucleic acid molecule; and
then identifying the carriers on the basis of the code.

There are many different variations to the subject methods including using the reporter molecule on a nucleic acid primer or nucleic acid binding agent as the carrier code. Furthermore, dividing the subject method into particular steps is not to impose any limitation as to the order of steps or the ability to combine two or more steps into a single step. For example, the codes may also be used to block free nucleic acid-binding sites on the particles.

In addition, in an alternate embodiment, the subject methods also contemplate the identification of binding partners of non-nucleic acid molecules by modification of the method above whereby a library comprising one or more non-nucleic acid molecules is immobilized to a nucleic acid encoded carrier, and putative binding partners, each labeled with a reporter molecule, are contacted with the immobilized molecule.

In a preferred alternate embodiment, the coded-carrier-immobilized molecules comprise a library of putative protein-binding molecules, which include but are not limited to putative enzyme inhibitors, and the binding partner is a protein or enzyme.

In relation to nucleic acid binding agents identified in accordance with the present invention, these extend to repressors, activators, expression modulators and nucleic acid binding agents for use, for example, as nucleic acid labeling reagents. Agents which activate gene expression or which repress gene expression are particularly preferred. General nucleic acid binding agents arc also useful for affinity chromatography and for nucleic acid purification.

The present invention further permits incorporation of the coded carriers with a nucleic acid anchoring system which facilitates ligase-mediated conjugation of a target nucleic acid molecule to the carrier via a tag oligonucleotide which is conjugated to the carrier via a covalent bond between a chemical moiety resident on the carrier and another chemical moiety on the tag nucleic acid molecule.

Another aspect of the present invention, therefore, is a tag oligonucleotide anchored to a carrier carrying a code distinctive of said carrier.

Accordingly, the present invention provides a carrier comprising a surface first chemical moiety capable of participating in covalent bond formation with a second chemical moiety conjugated to a tag oligonucleotide wherein the tag oligonucleotide is a substrate for ligase-mediated covalent bonding to a target nucleic acid molecule, said carrier further comprising a code distinctive of said carrier.

The chemical moiety on the surface of the carrier may be capable of covalent bond formation with an amine group, a thiol group or an acryl group.

Alternatively, the carrier surface moiety is selected from an amine group, a thiol group or an acryl group which is capable of covalent bond formation with a chemical moiety such as a carboxyl group on a tag oligonucleotide.

Accordingly, another aspect of the present invention is directed to a carrier comprising a surface first chemical moiety selected from a carboxyl group, an amine group, a thiol group and an acryl group, said first chemical moiety capable of participating in covalent bond formation with a second chemical moiety selected from a carboxyl group, an amine group, a thiol group and an acryl group conjugated to an oligonucleotide with the proviso that when the carrier surface moiety is a carboxyl group then the covalent bond forms with an amine group, a thiol group or an acryl group, said carrier further comprising a code distinctive of said carrier. The present invention extends, however, to chemical moieties capable of any form of covalent bond formation with any other chemical entity.

In one preferred embodiment, the chemical moiety on the surface of the carrier is a thiol group and such a group is capable of covalent bond formation with a number of chemical moieties such as one of an amine group, a thiol group or an acryl group and when the carrier chemical moiety is one of an amine group, a thiol group or an acryl group then the covalent bond is formed with a carboxyl group on the tag oligonucleotide.

In a most preferred embodiment, the carrier surface chemical moiety is a thiol group.

As indicated above, the carrier is preferably in the form of a solid support such as a microsphere, bead, glass, ceramic or plastic slide, a dipstick or the wall of a vessel such as a microtiter well. The form of the solid support is not critical and may vary depending on the application intended. However, microspheres such as silica or methacrylate microspheres are particularly useful in the practice of the present invention, especially for use in suspension arrays or optical fiber arrays.

The selection of solid supports is conveniently based on case of manipulation, level of expense, thermal stability and/or stability in aqueous and/or organic solvents.

In a particularly preferred embodiment, therefore, the present invention is directed to microspheres having a carboxylated or thiolated surface capable of participating in covalent bond formation with a chemical moiety selected from an amine group, a thiol group and an acryl group conjugated to a tag oligonucleotide.

Generally, any number of chemical moieties may be present or exposed on the surface of the solid support and these may range from a few hundred to several million.

In a particularly preferred embodiment, there are from about 3,000 to about 100,000 surface chemical moieties potentially involved in covalent bonding per carrier. This is particularly the case when the sold support is a microsphere. Conveniently, the microsphere comprises from about 4,000 to about 80,000 or more conveniently from about 5,000 to about 70,000 chemical moieties per bead.

In relation to one preferred embodiment, therefore, the present invention provides microspheres each comprising from about 3,000 to about 100,000 such as about 4,000 to about 80,000 or more particularly about 3,000 to about 5,000 surface carboxyl, or thiol groups or amine groups per microsphere.

The tag oligonucleotide having the chemical moiety capable of covalent bond formation with the carrier surface chemical moiety may comprise any nucleotide sequence although the nucleotide sequence would generally be known. One particularly useful sequence is an RNA polymerase promoter nucleotide sequence such as the SP6 RNA polymerase promoter nucleotide sequence. The benefit of the latter in terms of linking DNA is the ability to generate RNA transcripts. However, any oligonucleotide of known sequence may be employed. The term “oligonucleotide” is not to be viewed to any limiting extent and may comprise from about 10 base pairs (bp) to hundreds of bp.

It is convenient to ensure that after binding of the tag oligonucleotide to the carrier that the tag oligonucleotide does not exhibit interference with the carrier surface. Consequently, a spacer molecule is generally included between the chemical moiety and the 5′ end of the tag oligonucleotide. A spacer comprising carbon-based molecules such as having from about C₁ to about C₁₀₀ carbon atoms, more preferably from about C₁₀ to about C₅₀ and even more preferably from about C₁₈ to about C₃₆ is particularly useful.

The spacer may also be multiple repeats such as 2×C₁₈ spacers or 3×C₆ spacers. The length of the spacer is not critical and may be varied depending on the intended application.

Consequently, another aspect of the present invention contemplates an isolated tag oligonucleotide comprising a chemical moiety capable of covalent bond formation with a chemical moiety on the surface of a carrier, said first mentioned chemical moiety conjugated to said tag oligonucleotide via a carbon molecule having mCₙ carbon atoms wherein C is a carbon atom, n is the number of carbon atoms and m is the number of repeats of Cₙ molecules and is 1 or greater than 1.

Generally, n is from about 1 to about 100 and m is preferably 1 or from about 2 to about 10.

Conveniently, the total number of carbon atoms is from about 20 to about 50.

The spacer molecule is conveniently an alkyl, alkenyl or an alkynyl molecule. Preferably, the spacer is a linear non-branched hydrocarbon although any other molecule may be employed to separate the oligonucleotide from the surface of the solid support, including DNA.

The 5′ tag oligonucleotide chemical moiety is conveniently an amine group, a thiol group or an acryl group if the carrier surface chemical moiety is an carboxyl group. Alternatively, the 5′ chemical moiety is a carboxyl group and the carrier surface chemical moiety is one or more of an amine group, a thiol group and/or an acryl group. Acryl thiol groups are particularly useful.

In a most preferred embodiment, the 5′ chemical moiety on the tag oligonucleotide is an acryl group. In accordance with the above aspect of the present invention, the solid support is preferably a microsphere although any solid support may be employed.

Accordingly, another aspect of the present invention provides a carrier comprising a tag oligonucleotide anchored to the surface of said carrier via a covalent bond between a chemical moiety on the surface of the carrier and a chemical moiety conjugated to said tag oligonucleotide via a carbon atom (C) spacer having the structure mCₙ wherein n is the number of carbon atoms from about 1 to about 100 and m is the number of repeats of the Cn molecules and is from about 1 to about 10 wherein the carrier further comprises a code distinctive of said carrier.

As indicated above, the covalent bond is conveniently a carboxyl group covalently bonded to an amine, thiol or acryl group. Furthermore, the carbon atom containing molecule is preferably from about 20 to about 50 carbon atoms in length.

Consequently, another aspect of the present invention comprises an article of manufacture having the structure:-
S−mCₙ−[x₁x₂ . . . xₚ]
wherein:
S is a solid support;
C is a carbon atom
n is the number of carbon atoms and is from about 1 to about 100;
m is the number of repeats of the Cₙ moieties and is from about 1 to about 10; and
[x₁x₂ . . . xₚ] is a nucleotide sequence of nucleotides x₁x₂ . . . xₚ wherein each of x₁x₂ . . . xₚ may be the same or different and the nucleotide length, p, is from 5 to about 200.

In the above formation, the schematic “—” represents a covalent bond such as, for example, an amide bond

The oligonucleotide sequence. i.e. x₁x₂ . . . xₚ is any known sequence such as the SP6 RNA polymerase promoter. The oligonucleotide sequence may also comprise an additional nucleotide sequence having, for example, translation start signals, ribosome binding sites and an initial common triplet.

It is particularly convenient to ensure or to measure successful covalent attachment of the tag oligonucleotide sequence to the carrier. This can be accomplished by incorporating a complementary internally labeled oligonucleotide sequence. Conveniently, the internally labeled oligonucleotide sequence is complementary to the 5′ end of the anchored tag oligonucleotide sequence. The internal label may be any suitable label such as 6-FAM at its 3′ end. The 5′ end is generally phosphorylated.

Accordingly, another aspect of the present invention provides a carrier comprising a tag oligonucleotide of known sequence anchored thereto via a covalent linkage between a chemical moiety on the surface of the carrier and a chemical moiety conjugated to the tag oligonucleotide via a molecule of n carbon atoms wherein n is from about 1 to about 100, said carrier further comprising a second oligonucleotide sequence annealed by base pairing to a complementary nucleotide sequence on said first mentioned tag oligonucleotides resulting in an overhang at the 3′ end of either the tag oligonucleotide or its complementary oligonucleotide wherein the carrier further comprises a code distinctive of said carrier.

Preferably, the second oligonucleotide sequence comprises a label and is used to measure the success or otherwise of the covalent anchoring of the first oligonucleotide sequence to the carrier.

The preferred label is Cy5 or Alexa 647.

Preferably, the first oligonucleotide sequence overhangs at its 3′ end over the second oligonucleotide sequence.

As indicated above, the second oligonucleotide is labeled and, hence, it becomes a convenient assay for the success or otherwise of covalent attachment of the first oligonucleotide to the carrier. One skilled in the art will immediately recognize that there are many variations in order to determine the extent of covalent linkage and that the present invention should not be only limited to one particular means.

The essence of this aspect of the invention is a carrier having a first tag oligonucleotide attached thereto via covalent linkage between a first chemical moiety on the surface of the carrier (e.g. a carboxyl group) and a second chemical moiety conjugated to the first oligonucleotide via a spacer molecule of length mC, as defined above and a second tag oligonucleotide, optionally labeled with a reporter molecule capable of giving an identifiable signal, which anneals to complementary nucleotide sequences on the first oligonucleotide to provide, in a preferred embodiment, a 3′ overhang of the first tag oligonucleotide and wherein the 5′ end of the second tag oligonucleotide is phosphorylated.

The complementary oligonucleotide to the tag oligonucleotide is referred to herein as α-tag or the α-tag oligonucleotide.

The present invention provides, therefore, in one embodiment:-
(i) a carrier such as a microsphere, microchip or the sides of a well in a microtiter plate; and
(ii) a tag oligonucleotide having a chemical moiety conjugated to the oligonucleotide via a molecule of mCₙ carbon atoms as described above;
   wherein the chemical moiety on the oligonucleotide is in covalent bond formation with a chemical moiety on the surface of the carrier:
(iii) a code distinctive of said carrier.

The present invention further provides a kit comprising coded carriers for nucleic acid molecules. Conveniently, the kit is in compartmental form with a compartment adapted to contain the coded nucleic acid carriers. Other compartments may also be included each adapted to contain other reagents required for the practice of the method. In an alternative, the kit comprises separate compartments for carriers and codes.

The present invention further contemplates a computer program product for assessing the codes on individual or groups of carriers, the product comprising:-
(1) code that receives as input values, the code associated with a carrier;
(2) code that compares said carrier code to provide assessment of the identity of carriers from a reference database; and
(3) a computer readable medium that stores the codes.

Still another aspect of the present invention extends to a computer for assessing codes on carriers wherein said computer comprises:-
(1) a machine-readable data storage medium comprising a data storage material encoded with machine-readable data, wherein said machine-readable data comprise values for the identity of codes on carriers;
(2) a working memory for storing instructions for processing said machine-readable data;
(3) a central-processing unit coupled to said working memory and to said machine-readable data storage medium, for processing said machine readable data to compare said values to provide an assessment of the identity of codes from a reference database; and
(4) an output hardware coupled to said central processing unit, for receiving the results of the identity of the codes.

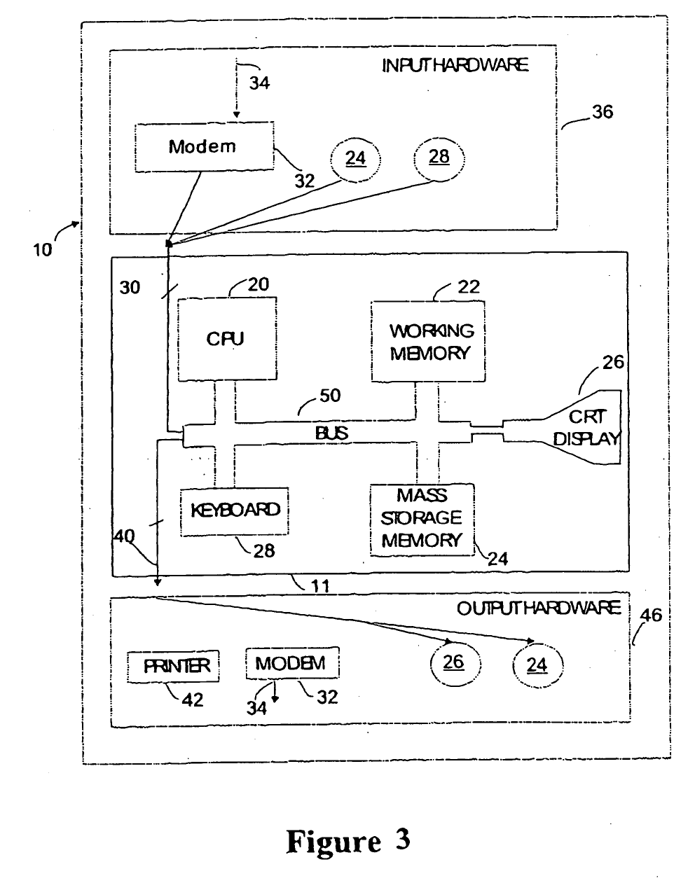

A version of these embodiments is presented in FIG. 3, which shows a system 10 including a computer 11 comprising a central processing unit (“CPU”) 20, a working memory 22 which may be, e.g. RAM (random-access memory) or “core” memory, mass storage memory 24 (such as one or more disk drives or CD-ROM drives), one or more cathode-ray tube (“CRT”) display terminals 26, one or more keyboards 28, one or more input lines 30, and one or more output lines 40, all of which are interconnected by a conventional bidirectional system bus 50.

Input hardware 36, coupled to computer 11 by input lines 30, may be implemented in a variety of ways. For example, machine-readable data of this invention may be inputted via the use of a modem or modems 32 connected by a telephone line or dedicated data line 34. Alternatively or additionally, the input hardware 36 may comprise CD. Alternatively, ROM drives or disk drives 24 in conjunction with display terminal 26, keyboard 28 may also be used as an input device.

Output hardware 46, coupled to computer 11 by output lines 40, may similarly be implemented by conventional devices. By way of example, output hardware 46 may include CRT display terminal 26 for displaying a synthetic polynucleotide sequence or a synthetic polypeptide sequence as described herein. Output hardware might also include a printer 42, so that hard copy output may be produced, or a disk drive 24, to store system output for later use.

In operation, CPU 20 coordinates the use of the various input and output devices 36, 46 coordinates data accesses from mass storage 24 and accesses to and from working memory 22, and determines the sequence of data processing steps. A number of programs may be used to process the machine readable data of this invention. Exemplary programs may use for example the following steps:-
(1) inputting input values for carriers based on the identity of the codes;
(2) assessing including comparing values for said nucleotide; and
(3) outputting the results of the assessment.

Generally, or conveniently, the codes are identified by mass and, hence, the computer product may interface with a mass spectrometer.

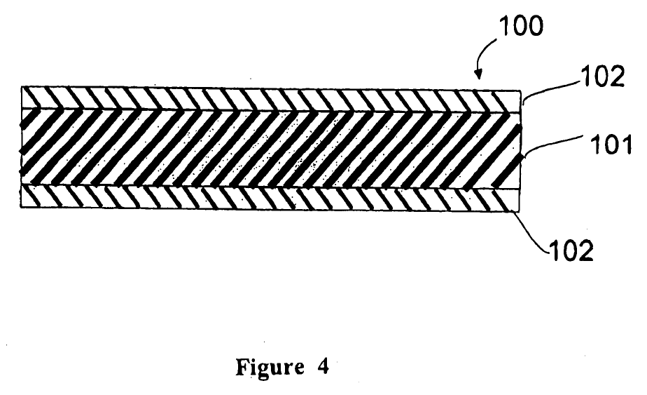

FIG. 4 shows a cross section of a magnetic data storage medium 100 which can be encoded with machine readable data, or set of instructions, for designing a synthetic molecule of the invention, which can be carried out by a system such as system 10 of FIG. 3. Medium 100 can be a conventional floppy diskette or hard disk, having a suitable substrate 101, which may be conventional, and a suitable coating 102, which may be conventional, on one or both sides, containing magnetic domains (not visible) whose polarity or orientation can be altered magnetically. Medium 100 may also have an opening (not shown) for receiving the spindle of a disk drive or other data storage device 24. The magnetic domains of coating 102 of medium 100 are polarized or oriented so as to encode in manner which may be conventional, machine readable data such as that described herein, for execution by a system such as system 10 of FIG. 3.

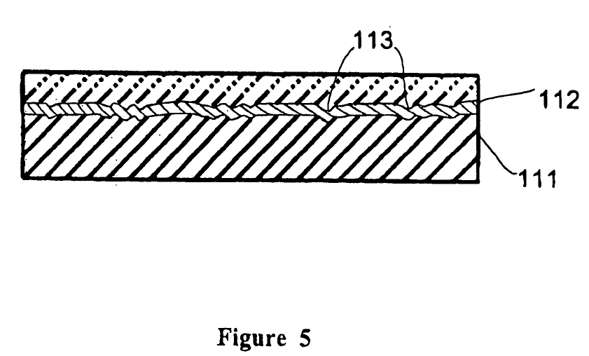

FIG. 5 shows a cross section of an optically readable data storage medium 110 which also can be encoded with such a machine-readable data, or set of instructions, for designing a synthetic molecule of the invention, which can be carried out by a system such as system 10 of FIG. 3. Medium 110 can be a conventional compact disk read only memory (CD-ROM) or a rewritable medium such as a magneto-optical disk, which is optically readable and magneto-optically writable. Medium 100 preferably has a suitable substrate 111, which may be conventional, and a suitable coating 112, which may be conventional, usually of one side of substrate 111.

In the case of CD-ROM, as is well known, coating 112 is reflective and is impressed with a plurality of pits 113 to encode the machine-readable data. The arrangement of pits is read by reflecting laser light off the surface of coating 112. A protective coating 114, which preferably is substantially transparent, is provided on top of coating 112.

The present invention is further described by the following non-limiting Examples.

### EXAMPLE 1

### Delection of Single Nucleotide Polymorphism

The Agouti signaling protein is associated with human pigmentation (Kanetsky et al., Am. J. Hum. Genet. 70: 770-775, 2002). A polymorphism is detected at genomic position 8818 A→G in the 3′ untranslated region of the ASIP gene. Carriage of the G allele was found to be associated with dark hair and brown eyes (Kanetsky et al., 2002, supra).

To screen a group of individuals for the polymorphisms, genomic DNA is amplified using primers which flank the 3′ untranslated region of the ASIP gene. The amplified DNA is then immobilized to microspheres labelled with a peptide code such that a separate code exists for each microsphere or group of microspheres carrying DNA from a single individual. Each separate peptide code is distinguishable on mass grounds.

An amplified reaction is then conducted using competitive primers, one corresponding to an A at 8818 and the other corresponding to a G at 8818. Suitable primers are disclosed by Kanetsky et al., 2002, supra. The two competitive primers are labelled with different fluorophores.

After amplification, an initial sorting using a flow cytometer bins the microspheres on the basis of incorporation of the fluorosphore. Mass spectrometry is then used to identify individual carriers based on the mass of codes.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### EXAMPLE 2

### Screening for Enzyme Inhibitors

Enzymes control many biological reactions such as glycolysis, power generation, signal transduction, etc. Many drugs work by inhibiting specific enzymatic reactions. To screen thousands of possible chemical inhibitors in a collection, or library, of small chemicals are conjugated to a specific DNA coded microsphere. The collection of microspheres is reacted to the enzyme to which a fluorescent tag has been attached.

After reaction with the enzyme:Fluor conjugate, the microspheres are subjected to sorting flow cytometry. Those beads which have bound the enzyme:fluor conjugate are sorted by fluorescence, most preferably into one microsphere per well. The chemicals are then determined by matching the sorted microspheres by DNA sequence on the bead. This is done in several ways. For example, Uracil N-glycosylase footprints of the DNA are determined by mass spectrometry. Alternatively, the DNA is first amplified by PCR or, if an RNA polymerase promoter is part of the DNA conjugate, by transcription. The cleavage patterns of the resulting mixture is deduced.

### EXAMPLE 3

### Dual Labelling of Silica Molecules

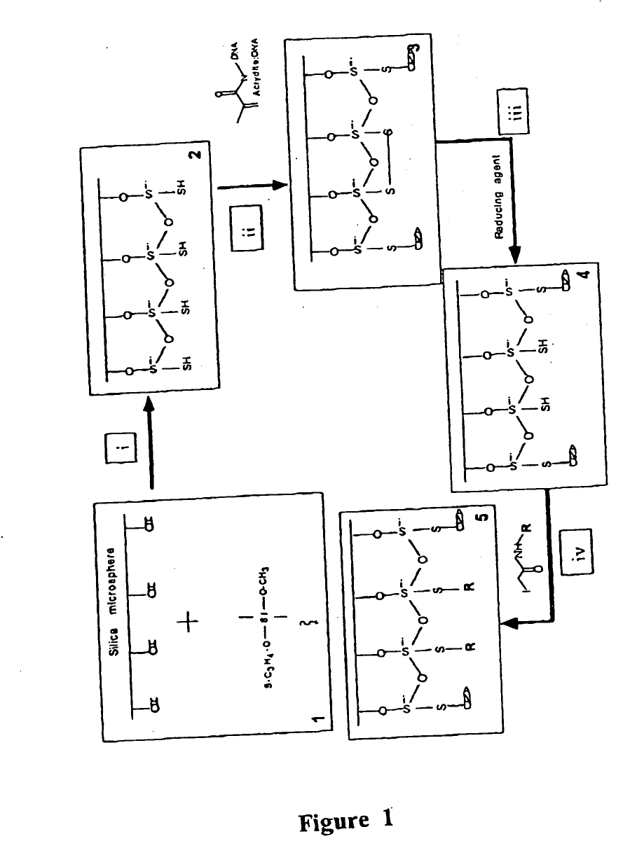

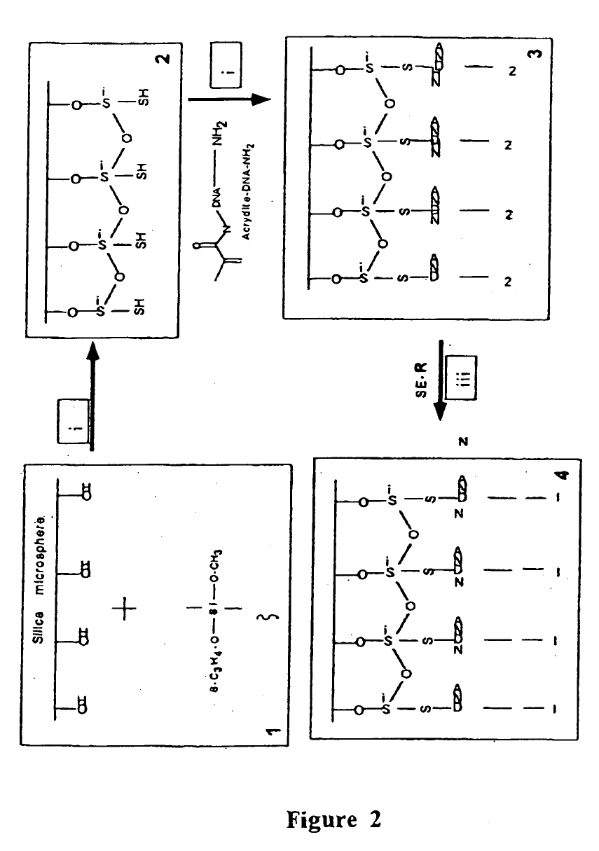

AmpaSand™ Beads labelled with DNA are co-labelled with specific chemicals using the following methods:
1. DNA is first bound to an activated surface by ethylene attack of surface thiols. The reaction is competed by Sulfur:Sulfur bonding between adjacent thiols. These S-S bonds are then reduced and thus reactivated for a second round of conjugation (see FIG. 1 for details).
2. The second method is similar with the modification that instead of reactivating thiols, a free amine carried as an internal modification of the DNA bound in the second step is used as the target in the second round conjugation. This scheme allows for any chemical compound reactive to amines to be attached covalently to the AmpaSand™ Bead (see FIG. 2 for details).

Interestingly, because of the covalent bond of the DNA to the silica surface, conjugations is carried out in virtually all organic solvents of moderate pH without disruption of the bead itself, or the bead:DNA thioether attachment.

Those skilled in the art will appreciate that the invention described herein is susceptible to variations and modifications other than those specifically described. It is to be understood that the invention includes all such variations and modifications. The invention also includes all of the steps, features, compositions and compounds referred to or indicated in this specification, individually or collectively, and any and all combinations of any two or more of said steps or features.

### BIBLIOGRAPHY

Biggs et al., Cytomerry 36: 36-45, 1999.
Caruso et al., J. Am. Chem. Soc. 120: 8523-8524, 1998.
Carzynkiewica and Crissman, Ed., “Methods in Cell Biology”, Vol. 33, Academic Press.
Daneshvar et al., J. Immunol. Methods 226(1-2): 119-128, 1999.
Dangl and Herzenberg, J. Immunol. Methods 52: 1-14, 1982.
Durig et al., J. Raman Spectrosc. 24(5): 281-285, 1993.
Eriksson et al., Biophys. J. 2: 64, 1993.
Fu et al., Nature Biotechnology 17: 1109-1111, 1999.
Gordon et al., J. Med. Chem. 37(10): 1385-1401, 1994.
Kanetsky et al., Am. J. Hum. Genet. 70: 770-775, 2002
Lakowicz et al., Biophys J. 72: 567, 1997.
Lewis et al., Dyes Pigm. 42(2): 197, 1999.
Malemed et al., “Flow cytometry and sorting”, 2^{nd} ed., New York, Wiley-Liss, 1990.
Rahman et al., J. Org. Chem. 63: 6196, 1998.
Ralph K. Iler, “The Colloid Chemistry of Silica and Silicates”, Cornell Universiiy Press, 1955.
Rapaport et al., Appl. Phys. Lett. 74(3): 329-331, 1999.
Shapiro, “Practical flow cytometry”, 3^{rd} Ed., Brisbane, Wiley-Liss, 1995.
Tawa et al., Mater. Res. Soc. Symp. Proc. 488: 885-890.
van Blaadern et al., Langinuir 8: 2921-2931, 1992.
van Helden and Vrij, Journal of Colloidal and Interface Science 76: 419-433, 1980.
Youvan et al., Biotechnology et Elia 3: 1-18, 1997.

1. A method for producing a plurality of carriers including a population of carriers having detectably distinct code molecules, said method comprising:
(i) preparing a plurality of carriers having different code molecules wherein each code molecule is associated with a respective carrier;
(ii) subjecting the nucleic acid molecules to nucleic acid-based reactions to enable incorporation of detectable labels into the immobilized nucleic acid molecules;
(iii) identifying carriers having distinctive code molecules that are detectably and/or quantifiably deciperable or resolvable by the detection/quantification means;
(iv) identifying carriers having similar to non-distinctive code molecules; and
(v) sorting carriers having distinctive code molecules from the carriers having non-distinctive code molecules to thereby provide a plurality of carriers including a population having detectably distinct code molecules.

2. The method of claim 1 wherein said nucleic acid molecules detectable label is attached via hybridization of a labeled primer or probe, optionally followed by amplification from said primer or probe.

3. The method of claim 1 or 2 wherein said detectable label is a fluorescent label.

4. The method of any one of claims 1 to 3 wherein sorting of said fluorescently labeled carriers according to the fluorescent label is performed using flow cytometry.

5. The method of any one of claims 1 to 4 wherein identification of said carriers having said distinctive code molecule is performed using mass spectroscopy.

6. The method of claim 1 wherein said molecule of each carrier is identified by an indirect or direct method for determining said nucleotide sequence of the nucleic acid molecule.

7. The method of claim 6 wherein said nucleotide sequence is determined using an assay selected from the group consisting of amplification of the sequence or part thereof, in-vitro transcription, restriction fragment length determination, automated or manual sequencing.

8. A carrier produced according to the method of any one of claims 1 to 7, said carrier comprising a solid or semi-solid support which is labeled with an identifiable code molecule that permits the differentiation of one such labelled carrier from another carrier in a heterogeneous population of said carriers.

9. The carrier of claim 8 wherein said immobilized molecule is a known nucleic acid molecule.

10. The carrier of any one of claims 8 or 9 wherein said code molecule is a peptide, that can be distinguished on the basis of molecular mass.

11. The carrier of any one of claims 8 to 10 wherein said carrier further comprises a chemical linking moiety which is capable of forming a chemical bond with a nucleic acid molecule.

12. The carrier of claim 11 wherein said chemical moiety comprises a thiol, carboxyl or amine group.

13. A nucleic acid molecule bound to or otherwise attached to said carrier of any one of claims 8 to 11.

14. The carrier of claim 8 wherein said immobilized molecule is a non-nucleic acid molecule and said code molecule is a nucleic acid which can be distinguished on the basis of nucleotide sequence.

15. The carrier of claim 13 wherein said immobilized molecule is a putative protein-binding molecule.

16. The carrier of claim 14 or 15 wherein said nucleic acid molecule is attached via a chemical linking moiety.

17. The carrier of claim 16 wherein said chemical moiety comprises a thiol, carboxyl or amine group.

18. The carrier of any one of claims 8 to 12 and 14 to 17 wherein said code or immobilized nucleic acid molecule is attached by a covalent bond between a chemical moiety on said surface of said carrier and a chemical moiety conjugated to said nucleic acid code molecule via a carbon atom spacer, having a structure mCn wherein n is the number of carbon atoms and is from 1 to about 100 and m is number of repeats of said Cn molecule and is from about 1 to about 10.

19. The carrier of any one of claims 8 to 12 and 14 to 18 wherein either said code or said immobilized nucleic acid code molecule may be transcribed and/or comprises an RNA polymerase promoter sequence or functional fragment thereof.

20. The carrier of claim 19 wherein the RNA polymerase promoter sequence is an SP6 RNA polymerase promoter sequence of functional fragment, homolog, analog, derivative thereof.

21. The carrier of any one of claims 8 to 12 and 14 to 20 wherein said solid or semi-solid support is a microparticle.

22. A population of carriers of any one of claims 8 to 12 and 14 to 20 wherein said population comprises one or more distinct classes of carrier on the basis of the attached code molecule.

23. A method for simultaneously detecting nucleotide polymorphisms in two or more subjects, said method comprising: amplifying or otherwise isolating a potentially polymorphic genetic sequence from two or more subjects in a population; binding the resultant polynucleotides from each subject to a uniquely coded carrier of any one of claims 8 to 12; competitively hybridizing one or more differentially labeled probes or primers to the carrier bound nucleic acid, wherein each probe or primer is specific for a polymorphic variant; optionally performing an amplification reaction primed from the bound primer(s) or probe; sorting the population of carriers according to the bound label; and identifying a particular carrier present in each distinct labelled group on the basis of the molecular mass of the code molecule; thereby associating particular subjects with a particular polymorphic sequence variant.

24. A method for identifying small molecule ligands of a protein, said method comprising: producing or acquiring a library of putative ligands of the protein of interest; attaching each member of the library to a differentially coded carrier of any one of claims 14 to 20; contacting the population of carriers with a labeled protein; sorting of the population by presence or absence of the bound label; and identification of the carriers that bind the subject protein by elucidation of the nucleotide sequence of the nucleic acid code; and thereby identification of a ligand of the protein by association of a library member with a particular code.

25. The method of claim 23 or 24 wherein said carrier comprises a microsphere.

26. The method of any one of claims 23 to 25 wherein said label is a fluorescent label.

27. The method of any one of claims 23 to 26 wherein said carriers are sorted using flow cytometry and/or Fluorescence-Assisted Cell Sorting (FACS).

28. A kit comprising one or more coded carriers of any one of claims 8 to 12 and 13 to 22 in compartmental form, said kit comprising one or more compartments containing one or more coded carriers, and optionally one or more compartments containing other reagents necessary for the use of said coded carriers, together with instructions for the use of said carriers.

29. A computer program product for assessing the codes on individual or groups of coded carriers according to any one of claims 8 to 22, the product comprising:
(i) code that receives as input values, the code associated with a carrier;
(ii) code that compares said carrier code to provide assessment of the identity of carriers from a reference database; and
(iii) a computer readable medium that stores the codes.

30. A computer for assessing codes on carriers according to claims 8 to 22, wherein said computer comprises:-
(i) a machine-readable data storage medium comprising a data storage material encoded with machine-readable data, wherein said machine-readable data comprise values for the identity of codes on carriers;
(ii) a working memory for storing instructions for processing said machine-readable data;
(iii) a central-processing unit coupled to said working memory and to said machine-readable data storage medium, for processing said machine readable data to compare said values to provide an assessment of the identity of codes from a reference database; and
(iv) an output hardware coupled to said central processing unit, for receiving the results of the identity of the codes.
